(19) United States
(12) Patent Application Publication
(10) Pub. No.: US 2006/0105938 A1
(43) Pub. Date: May 18, 2006

(54) Method of treating or preventing obeisity and lipid metabolism disorders and compositions for use therein

(76) Inventors: Andries Siemensma, Le Leeuwarden (NL); Anthonie Kunst, An Huizen (NL); Marcellinus Hakkaart, Vn Hilversum (NL)

Correspondence Address: BIRCH STEWART KOLASCH & BIRCH, PO BOX 747, FALLS CHURCH, VA, 22040-0747, US

(21) Appl. No.: 10/527,754

(22) PCT Filed: Sep. 16, 2003
(86) PCT No.: PCT/NL03/00641

§ 371 Date: Nov. 3, 2005

(30) Foreign Application Priority Data
Sep. 16, 2002 (NL) 02078811.3

(51) Int. Cl. A61K 38/16; A61K 31/70

(52) U.S. Cl. 514/2; 514/23

(57) ABSTRACT

The present invention provides a method of preventing or treating human obesity, said method comprising ingesting a composition containing, calculated on dry matter: 10-100 wt. % protein hydrolysate; 0-90 wt. % intact protein; 0-50 wt. % carbohydrate; and wherein hydrolysed protein and intact protein together are present in a concentration (w/w) that exceeds the carbohydrate concentration (w/w). The invention also encompasses the use of the same composition in a method of preventing or treating lipid metabolism disorders and in a method for improving body appearance. Other aspects of the invention relate to nutritional beverages, snacks and soups that can advantageously be employed in accordance with the aforementioned methods.

### TECHNICAL FIELD OF THE INVENTION

The present invention relates to a method for preventing or treating obesity as well as for preventing or treating lipid metabolism disorders. Also provided herein is a cosmetic method for improving body appearance.

In addition, the present invention concerns compositions for use in the aforementioned methods. More particularly, the invention provides nutritional beverages, snacks and soups for use in said methods.

### BACKGROUND OF THE INVENTION

Obesity is a risk factor associated with various adult diseases such as diabetes type 2 and cardiovascular diseases including hypertension. It may also be a cause for deteriorating these diseases. Both adults and children may face the problem of obesity. Currently, about 25% of the children and 50% of the adults in the USA are overweight, prevalence rates that have increased by 50% since the 1960s. The obesity in children has been associated with consumption of sugar-sweetened drinks.

Lipid metabolism disorders are a well-known complication of obesity. These disorders are often characterised by hyperinsulinaemia, elevated apolipoprotein B levels, high triglycerides concentration, the presence of small dense LDL, high LDL cholesterol concentration, and low HDL cholesterol concentration.

With the trend of healthy eating, also the trend of being fit or stay lean has emerged. Accordingly, not only persons affected by obesity are concerned with the problem of controlling or losing weight, but also healthy individuals, individuals who do not suffer from obesity often have a desire to stay lean or to lose weight in order to obtain a cosmetically more acceptable or more desirable appearance.

The growing concern of the human population about overweight has resulted in an increasing intensity of research in this field. Recently, Jequier concluded (“pathways to obesity” Int J Obes Relat Metab Disord 2002; 26 (suppl):2:S12-7) that the prevalence of obesity had reached epidemic proportions in affluent societies and indicated that the primary cause of obesity was lying more in environmental and behavioural changes than in genetic modifications.

It has been tried to solve the problem of overweight by using of medication. This approach, however, suffers from the disadvantage that after a period of about 3 months patients seem to reach a plateau as far as weight loss is concerned. Also, the medication does not diminish the “hungry” feeling, i.e. it fails to impart satiety. Another disadvantage of the use of medication is the potential for side effects, which sometimes outweighs the benefits of taking medication.

Accordingly, studies were done to find a more natural solution. One simple natural solution is to exercise. However, to obtain a noticeable result, exercising on a regular basis is a pre-requisite that, with today's trends of habits, is not always feasible. Another simple natural solution is to eat less or to adapt the diet. Remedies based on consumption of low-fat diets have been found to produce some result. On the short-term low-fat diets induce a modest weight loss in obese individuals but on the longer term (more than a year) such diets appear to have little effect on body fatness.

Many low fat commercial food products are based on the recommendations of the American Hearth Association (AHA) and derive a low percentage of energy from fat and a high percentage from carbohydrate. Despite the popularity of these products and the reduction (on population base) of the percentage of dietary energy derived from fat, the prevalence of obesity has continued to increase. This clearly suggests that fat is only one of several determinants and not the primary cause of the high prevalence of excess body fat in our Western society. (“Is dietary fat a major determinant of body fat?”, Am. J. Clin. Nutr. 1998; 67(suppl):556S-62S.).

Another dietary factor that may play a critical role in body weight regulation is the glycaemic index (GI). The concept of the GI was proposed by Jenkins et all (“Glycemic index of foods: a physiological basis for carbohydrate exchange”. Am J Clin Nutr 1981; 34:362-366), to characterize the rate of carbohydrate absorption after a meal. It is defined as the area under the glucose response curve after consumption of 50 g carbohydrate from a test food divided by the area under the curve after consumption of 50 g carbohydrate from a control food, either white bread or glucose. Many factors together, including carbohydrate type, fibre, protein, fat, food form and method of preparation, determine the GI of a particular food.

Recently a new method of dieting called the “Montignac method” has been described. This method involves balancing the administration of protein and carbohydrate. In particular, the Montignac method is an ad libitum low GI/low fat/high protein diet in which carbohydrates with a glycaemic index above 55 are excluded.

In the underlying physiological mechanism insulin is believed to play an important role. Insulin stimulates the uptake of glucose from the blood and subsequent conversion of glucose into the storage carbohydrate glycogen or into lipids when glycogen storage is saturated.

It is well known that amino acids can stimulate the release of insulin upon a glucose load. A rapid rise of the glucose level in the blood induces a quick increase in the insulin level, resulting in a rapid fall of the blood glucose level. This induces a feeling of craving or hunger and urges someone to eat. It is speculated that this phenomenon plays an important role in the weight gain observed in diets based on low fat and high carbohydrates.

Although the Montignac method has shown good results, people have found it difficult to follow it on a long-term basis, especially once the weight loss is obtained. As a result, the benefits obtained are often lost within a few weeks after discontinuation of the method.

### SUMMARY OF THE INVENTION

It has now been found that repeated ingestion of a composition containing at least 10% protein hydrolysate from a non-mammalian source by dry weight, provides a natural solution to the problem of losing weight as well as to the problem of lipid metabolism. This effect is especially seen upon long term use of the composition. The present composition is characterised in that it optionally contains intact protein and/or carbohydrate, provided hydrolysed protein and intact protein together are present in a concentration (w/w) that exceeds the carbohydrate concentration (w/w). The presence of high levels of carbohydrate, relative to the total content of non-mammalian hydrolysed protein and intact protein, is likely to produce an undesired insulin response that will counteract the desired impact of the present method.

In one aspect of the invention, there is provided a method for preventing and/or treating human obesity, said method comprising ingesting a composition, containing, calculated on dry matter:

10-100 wt % protein hydrolysate;

0-90 wt. % intact protein;

0-50 wt. % carbohydrate; and

wherein hydrolysed protein and intact protein together are present in a concentration (w/w) that exceeds the carbohydrate concentration (w/w).

In another aspect of the invention, there is provided a method of preventing or treating lipid metabolism disorders, said method comprising ingesting the compositions as defined above.

In another aspect of the invention, there is provided a cosmetic method for stimulating and/or improving body weight reduction in order to improve the human body appearance, said method omprising ingesting the composition as defined herein before.

A further aspect of the invention relates to a nutritional beverage containing:

0.5-10 wt. % hydrolysed protein from a non-mammalian source;

0-10 wt. % intact protein;

0.5-5 wt. % carbohydrates;

artificial sweetener;

flavouring; and

at least 80 wt. % water;

wherein hydrolysed protein and intact protein together are present in a concentration (w/w) that exceeds the carbohydrate concentration (w/w).

Yet another aspect of the invention concerns a savoury snack product containing:

3-50 wt. % hydrolysed protein from a non-mammalian source;

10-50 wt. % intact protein;

0-50 wt. % carbohydrate;

0.3-3 wt. % salt; and

0.1-20 wt. % water,

wherein hydrolysed protein and intact protein together are present in a concentration (w/w) that exceeds the carbohydrate concentration (w/w).

Finally, the present invention also encompasses a soup containing:

0.5-20 wt. % hydrolysed protein from a non-mammalian source;

0.5-30 wt. % intact protein;

0-20 wt. % carbohydrate;

0.1-2 wt. % salt; and

at least 70 wt. % water;

wherein hydrolysed protein and intact protein together are present in a concentration (w/w) that exceeds the carbohydrate concentration (w/w).

### DETAILED DESCRIPTION OF THE INVENTION

A—Protein Hydrolysate

The use of a protein hydrolysate from a non-mammalian source is an essential element of the present invention. Indeed, the use of a composition containing at least 10% of this ingredient by weight of dry matter has been found beneficial for patients suffering from obesity, but also for healthy persons wanting to stay lean. Accordingly, the use of such a protein hydrolysate has been found effective in the prevention and treatment of human obesity. Advantageously, it was also found to be effective in the prevention and treatment of lipid metabolism disorders in humans, in particular by reducing LDL cholesterol concentration and/or reducing the triglycerides concentration and/or increasing HDL cholesterol concentration. Preferably, such treatment results in an improvement of all three parameters.

Yet, another group of persons that can derive benefit from ingesting protein hydrolysates from a non-mammalian source are healthy individuals who have a desire to stay lean. The term “healthy individuals” as used in here refers to individuals not suffering from obesity.

The term “non-mammalian proteins” refers to proteins that are not obtained from mammalian sources. Accordingly, non-mammalian proteins include proteins from the non-limiting sources: vegetable proteins, fungal proteins, microbial proteins, fish proteins, poultry proteins, egg proteins, as well as mixtures thereof.

The term “protein hydrolysate” is used to define a protein raw material that has been hydrolysed by one or more hydrolytic enzymes. The hydrolytic enzyme can be of animal, plant, yeast, bacterial or fungal origin. Preferably enzyme preparations are used which have a low exo-peptidase activity to minimise the liberation of free amino acids and to improve taste profiles of the protein hydrolysates. The preferred hydrolysed protein material of the present invention has an average peptide chain length in the range of 2-40 amino acid residues and more preferably in the range of 3-20 amino acid residues. The average peptide chain can be determined using the method as described in WO 96/26266. The protein hydrolysates that can be used to prepare a composition as disclosed in the present invention are not limited to ones disclosed in the present invention but include all protein hydrolysates that can be obtained by enzymatic hydrolysis using common techniques as described in the literature and known to those skilled in the art.

Preferably, the non-mammalian protein hydrolysates used in accordance with the present invention are obtained from a protein selected from vegetable proteins, fungal proteins, microbial proteins, fish proteins, poultry proteins, egg proteins, and mixtures thereof.

Vegetable protein hydrolysates are hydrolysates of vegetable protein obtained from protein selected from wheat, maize, pea, rice, soy, barley, oats, potato, and mixtures thereof, more preferably the vegetable protein hydrolysate is a pea protein hydrolysate, a rice protein hydrolysate or a soy protein hydrolysate. Most preferably, the hydrolysate is a pea protein hydrolysate.

For the purpose of the present invention, the vegetable protein hydrolysates are preferred. Indeed, it has been found that the vegetable protein hydrolysate, especially the hydrolysates obtained from pea protein, rice protein and/or soy protein, best influenced the weight reduction in obese people on a hypocaloric diet.

Ingestion of a composition containing at least 10% of the protein hydrolysate of a non-mammalian source by weight of dry matter induces a long lasting feeling of satiety (which stops people from eating even in ad libitum situations) without giving rise to a substantial increase in the blood insulin level (which urges people to eat), even if the composition also contains a significant amount of carbohydrate. This is related to the high solubility of the present protein hydrolysate, which allows it to reach the intestinal tract quickly and in a relatively high concentration. It was observed that, even in situations where food is available ad libitum, individuals consume less in the hours after ingesting the present composition containing the non-mammalian protein hydrolysate. This obviously has positive implications for the ability to control body weight.

The non-mammalian protein hydrolysate is preferably incorporated in a composition comprising one or more materials selected from the group consisting of carbohydrates, intact proteins (both mammalian and non-mammalian), free amino acids, minerals, vitamins, dietary fibers, herbals, spices, flavors, fat, and mixtures thereof. Preferably, the composition comprises a carbohydrate, more preferably together with materials selected from the group consisting of intact proteins, free amino acids, minerals, vitamins, dietary fibers, herbals, spices, flavors, fat, and mixtures thereof.

B—Methods of Preventing or Treating Obesity or Lipid Metabolism Disorders

The present methods employ a composition that may optionally contain carbohydrate, provided the amount of carbohydrate does not exceed the combined amount of non-mammalian protein hydrolysate and intact protein contained within the same composition. In a particularly preferred embodiment, the present composition contains proteinaceous matter and carbohydrate in a weight ratio within the range of 1.5:1 to 9:1, most preferably within the range of 1.5:1 to 4:1. For the purpose of this ratio calculation, the term “proteinaceous matter” includes the present protein hydrolysate from a non-mammalian source as well as free amino acids and intact protein material.

The term “intact protein” refers to proteins that have not been subjected to modifications such as hydrolysis. The term “free amino acid” refers to amino acids per se, i.e. said term does not include amino acid residues contained in indirect sources like proteins.

The benefits of the present composition are particularly pronounced in case the composition contains the protein hydrolysate from a non-mammalian source in a higher amount than the optional carbohydrate component. In a particularly preferred embodiment, the composition contains the protein hydrolysate and the optional carbohydrate in a weight ratio of more than 1:3, more preferably in a weight ratio of more than 1:1.

The present composition preferably does not contain substantial amounts of carbohydrate as excess amounts of carbohydrate may lead to undesirable insulin surges, despite the tempering effect of the non-mammalian protein hydrolysate. In a preferred embodiment, the composition contains 0-30% carbohydrate by weight of dry matter.

The present composition advantageously contains intact protein. Intact protein is suitably incorporated as an energy source and to improve the palatability of the composition. Typically, the composition contains 20-80% intact protein by weight of dry matter.

The present composition may suitably contain some carbohydrate material. Indeed, by selecting the right type(s) of carbohydrate, it was found that the insulin response, believed to be an important factor in weight gain, was neglectable, even after a few weeks of dieting with the present composition.

On the basis of their rate of release of glucose into the blood, carbohydrates can be divided into three classes (Cummings J H and Englyst H N, AJCN 1995; 61 (4 Suppl):938S-945S). The carbohydrate of the invention is advantageously selected from at least one member of these classes:

a) Rapidly Absorbed Carbohydrates

This class comprises carbohydrates such as glucose and disaccharides containing a glucose unit such as sucrose, lactose, maltose and galactose that contribute directly to elevation in blood glucose. Sucrose, in addition to being rapidly absorbed, has the advantage of imparting a sweet taste to the composition thereby increasing palatability.

To keep the insulin response low after consumption of these type of carbohydrates it is preferred to limit the intake to not more than 6, preferably not more than 5 gram per serving or per moment of eating.

To limit the intake of the rapidly absorbed carbohydrates to 6 gram for a beverage with a typical serving of 200 ml, the concentration therefore should not exceed 6 gram per 200 ml (3% w/v). In beverages for which the typical serving is larger the concentration should be accordingly lower. A snack, such as a nutritional bar, typically weighs 60 grams. In order to limit the intake of the rapidly absorbed carbohydrates in such a snack to not more than 6 gram, the concentration should not exceed 6 gram per 60 gram (10% w/w).

b) Moderately Absorbed Carbohydrates

This class comprise mono- and disaccharides, that do not contribute directly to elevation of blood glucose as well as those soluble and insoluble polysaccharides (e.g. starches) that contain at least 30 molar % glucose units and that release a majority of this glucose upon incubation in pancreatic amylase and amyloglucosidase at 37° C. in 20 minutes or less.

The monosaccharides and disaccharides that are considered moderately absorbed are non-glucose monosaccharides and non-glucose-containing disaccharides that contribute to blood glucose levels indirectly, i.e., after a metabolic event occurs, e.g., conversion into glucose by the liver. Non-limiting examples of such moderately absorbed carbohydrates are mannose and fructose. The class of moderately absorbed carbohydrates also includes polysaccharides that contain glucose units and have a dextrose equivalent of 15 or lower, such as white flour, wheat flour, certain starches, and the like.

To keep the insulin response low after consumption of these type of carbohydrates it is preferred to limit the intake to not more than 20 gram per serving or per moment of eating.

c) Slowly Absorbed Carbohydrates

This class comprises polysaccharides (carbohydrates containing three or more monomeric units) that contain at least 30 molar % glucose units, have a glycaemic index greater than 2, and release a majority of their glucose in greater than 20 minutes upon incubation in pancreatic amylase and amyloglucosidase at 37° C. Non-limiting examples of slowly absorbed polysaccharides include corn starch, high amylose corn starch (e.g. corn starch with an amylose content of greater than 40% by weight) and modified starches with a glycaemic index less than 80, preferably of less than 60. Examples of such products are Novelose, Systain 550 and 735 from National Starch.

Accordingly, the carbohydrate of the invention is advantageously selected from at least one member of the class consisting of:

a) a rapidly absorbed fraction comprising components selected from glucose, sucrose, one or more rapidly absorbed disaccharides containing a glucose unit, and mixtures thereof;

b) a moderately absorbed fraction comprising components selected from one or more moderately absorbed monosaccharides, disaccharides, glucose-containing polysaccharides, and mixtures thereof;

c) a slowly absorbed fraction comprising components selected from one or more slowly absorbed glucose-containing polysaccharides.

The carbohydrates incorporated in the present composition have to be selected carefully in order to ensure that ingestion of the composition will not be accompanied by major insuline surges in the blood. Preferably, to this end, at least a sizeable fraction of the carbohydrates should be slowly released and/or slowly digested such that the release of glucose into the blood is slow.

In case the present composition contains carbohydrate, it is preferred that at least a fraction of said carbohydrate is slowly absorbed carbohydrate. The slowly absorbed fraction of the carbohydrate component typically constitutes about 1 to about 95 weight (wt) % of total carbohydrate component, preferably about 5 to about 85 wt %, and more preferably about 20 to about 75 wt %.

The present composition may suitably contains rapidly and/or moderately absorbed carbohydrates. Preferably, the composition contains between 0.5 and 5% rapidly and/or moderately absorbed carbohydrates by weight of dry matter. The rapidly absorbed fraction of the carbohydrate component typically constitutes about 1 to about 95 weight (wt) % of total carbohydrate component, preferably about 5 to about 85 wt %, and more preferably about 20 to about 75 wt %. The moderately absorbed fraction of the carbohydrate component typically constitutes about 1 to about 95 weight (wt) % of total carbohydrate component, preferably about 5 to about 85 wt %, and more preferably about 20 to about 75 wt %.

In a particularly preferred embodiment, a major part of the carbohydrates contained in the present composition are slowly absorbed carbohydrates. Even more preferably, the composition preferably contains slowly absorbed carbohydrates in a concentration (w/w) that exceeds the combined concentration (w/w) of rapidly and moderately absorbed carbohydrates by at least a factor 2.

Preferred carbohydrates are carbohydrates wherein the rapidly absorbed fraction is selected from glucose, sucrose, maltose, and mixtures thereof, the moderately absorbed fraction is selected from fructose, mannose, maltodextrin, white flour, wheat flour, and mixtures thereof, and the slowly absorbed fraction is selected from raw corn starch, high amylose corn starch, modified starch, and mixtures thereof.

For solid or semi-solid compositions the slowly absorbed polysaccharide preferably comprises high amylose corn starch, modified starch (as described above), or a mixture thereof A preferred slowly absorbed carbohydrate is Novelose resistant starch, which is a high amylose corn starch available from National Starch. For liquid products, raw corn starch is preferred.

The composition of the invention optionally contains one or more flavours including natural or artificial flavourants to enhance palatability such as herbals, spices, flavour ingredient. Any flavourant used in the art can be included such as strawberry; cherry; chocolate; orange; citrus; lemon; grapefruit; coconut; vanilla; spices such as nutmeg, cinnamon and the like.

The composition may suitably contain a fat component. The fat component can be any lipid or fat known in the art to be suitable for use in nutritional compositions. Typical fats include milk fat, safflower oil, canola oil, egg yolk lipid, olive oil, cotton seed oil, coconut oil, palm oil, palm kernel oil, soybean oil, sunflower oil, fish oil and fractions of all above oils derived thereof such as palm olein, medium chain triglycerides (MCT), and esters of fatty acids wherein the fatty acids are, for example, arachidonic acid, linoleic acid, palmitic acid, stearic acid, docosahexaeonic acid, eicosapentaenoic acid, linolenic acid, oleic acid, lauric acid, capric acid, caprylic acid, caproic acid, and the like. High oleic forms of various oils are also contemplated to be useful herein such as high oleic sunflower oil and high oleic safflower oil. Preferably the fat level in the composition is such that less than 40% of the energy in the composition is provided by fat.

The composition employed in the present methods may suitably be free from carbohydrates, especially if said product is intended to be used as a nutraceutical. Alternatively, said composition may be a food product that meets the compositional requirements described herein before. Such food products, in addition to the present protein hydrolysate, typically will contain intact protein, carbohydrates and optionally fat. Modified food products that are particularly suited for use in the present method include beverages, snacks and soups. The latter products are suitably consumed between meals. Thus, by ingesting such a product between 0.5 and 2 hours prior to the next meal, a feeling of satiety will be induced that will persist throughout the meal, leading to less caloric intake.

The benefits of the present method will become particularly apparent in case the method is continued uninterruptedly for at least 2 weeks, more preferably at least 4 weeks, most preferably at least 3 months. Typically, the method according to the invention comprises at least once daily ingestion of the present composition. More preferably, said composition is ingested at least twice daily.

Typically, the present method comprises ingesting the present composition in an amount equivalent to at least 5 g, preferably at least 10 g of the combination of protein hydrolysate and intact protein per serving. Most preferably, the aforementioned minimum amounts relate to the amount of non-mammalian protein hydrolysates that are ingested per serving.

### C)—PRODUCTS SUITABLE FOR USE IN THE PRESENT METHOD

The method described herein before may suitably employ a composition in the form of a food products, especially a food product selected from a nutritional beverage, a snack and a soup.

Accordingly, one aspect of the present invention relates to a nutritional beverage containing:
0.5-10 wt. %, preferably 0.8-6 wt. %, more preferably 1-4 wt. % hydrolysed protein from a non-mammalian source;
0-10 wt. %, preferably 1-9 wt. % intact protein;
0.5-5 wt. % carbohydrates, preferably rapidly absorbed carbohydrates;
artificial sweetener;
flavouring; and
at least 80 wt. % water;
   wherein hydrolysed protein and intact protein together are present in a concentration (w/w) that exceeds the carbohydrate concentration (w/w).

The aforementioned beverage is particularly suited for use in the present method because it can be consumed conveniently at any moment during the day. Also this embodiment offers the advantage that it is feasible to formulate a pleasant tasting low caloric beverage that can suitably be used to deliver a large amount of protein hydrolysate. In a preferred embodiment, hydrolysed protein and intact protein together represent at least 5 wt. % of the beverage.

Another aspect of the invention is concerned with a savoury snack product containing:
3-50 wt. %, preferably 4-30 wt. %, more preferably 5-20 wt. % hydrolysed protein from a non-mammalian source;
10-80 wt. %, preferably 20-70 wt. % intact protein;
0-50 wt. % carbohydrate; preferably 0-30 wt. % carbohydrate;
0.3-3 wt. % salt; and
0.1-20 wt. % water;
   wherein hydrolysed protein and intact protein together are present in a concentration (w/w) that exceeds the carbohydrate concentration (w/w).

The present snack product may suitably take the form of a nutritional bar or a baked or fried product. In a particularly preferred embodiment, the snack product contains 1-20 wt. % dietary fibre.

Yet another aspect of the invention relates to a soup containing:
0.5-20 wt. %, preferably 1-20 wt. %, more preferably 2-20 wt. % hydrolysed protein from a non-mammalian source;
0.5-29 wt. %, preferably 1-25 wt. % intact protein;
0-20 wt. % carbohydrate;
0.1-2 wt. % salt; and
at least 70 wt. % water,
   wherein hydrolysed protein and intact protein together are present in a concentration (w/w) that exceeds the carbohydrate concentration (w/w).

The composition and products according to the present invention may suitably comprise one or more optional ingredients selected from intact proteins, free amino acids, minerals, vitamins, dietary fibers, herbals, spices, flavors, fat, and mixtures thereof. Typical examples of minerals, vitamins and other nutrients that may be employed in the present composition and products include vitamin A, vitamin B6, vitamin B12, vitamin E, vitamin K, vitamin C, vitamin D, inositol, taurine, folic acid, thiamine, riboflavin, niacin, biotin, pantothenic acid, choline, calcium, phosphorous, iodine, iron, magnesium, copper, zinc, manganese, chloride, potassium, sodium, carotenoids, flavonoids, lipoic acid, nucleotides, selenium, chromium, molybdenum, and L-carnitine. Minerals are usually added in salt form.

Typical dietary fibers include fibers and non-absorbant carbohydrates that have a glycaemic index less than 2. The fiber can be soluble, insoluble, fermentable, non-fermentable, or any combination thereof. The fiber can be, for example, soy fiber, pectin, certain resistant starches, oligofructose, inulins, oat fiber, pea fiber, guar gum, gum acacia, modified cellulose, and the like.

### D)—EXAMPLES

The present invention is illustrated in the non-limiting following examples:

### Example 1

### Spiced Gingerbread

The following is the composition of a spiced gingerbread incorporating the invention, which ingredients were mixed into a homogeneous batter prior to being baked in an oven for 75 min at 150° C. The list of ingredient is as follows:

50 gram wheat gluten

125 gram wheat starch

45 gram fructose

150 gram Systain® 735 (National Starch)

15 gram biscuit spices

10 gram cinnamon

300 ml sugar free fruit syrup

15 gram baking powder

400 ml water

375 gram pea protein hydrolysate Hyprol™ 7102 (Quest International)

300 gram apple pieces

### Example 2

### Soda Drink

A protein-rich drink was prepared by adding 9.16 grams per 100 ml of the powder composed out of the following ingredients:

| | |
|---|---|
| Hydrolysed pea protein (agglomerated Hyprol ™ 7102 Dev) | 7.50 g |
| Low GI carbohydrate (Systain ® 550) | 1.20 g |
| Citric acid | 0.20 g |
| Malic acid | 0.10 g |
| Artificial sweeteners (aspartame and acesulfame) | 0.04 g |
| Flavour compounds | 0.12 g |

### Example 3

### Protein Drink

A protein-rich drink was prepared by adding 9.05 grams per 100 ml of the powder comprising the following ingredients:

| | |
|---|---|
| Pea protein isolate | 5.88 g |
| Hydrolysed pea protein (Hyfoama ™ PW) | 1.30 g |
| Low GI carbohydrate (Systain ® 735 | 1.20 g |
| Artificial sweetener (acesulfame, aspartame) | 0.05 g |
| Stabilizer | 0.20 g |
| Flavour compounds | 0.42 g |

### Example 4

### Sheeted Snack

A protein-rich sheeted snack was prepared by adding to 60 ml of water 100 grams of a powder containing following ingredients:

| | |
|---|---|
| Pea powder | 20 g |
| Pea protein isolate | 57 g |
| Pea hydrolysate Hyprol ™ 7102 Dev | 10 g |
| Bakasnack | 5 g |
| Polyglycerolester | 1 g |
| Dietary fiber | 5 g |
| Salt | 2 g |

The ingredients were mixed into a homogeneous batter prior to being sheeted and deep-fried.

1-19. (canceled)

20. A method of preventing and/or treating human obesity, said method comprising ingesting a composition containing, calculated on dry matter:
10-100 wt % protein hydrolysate originating from a non-mammalian source;
0-90 wt. % intact protein;
0-50 wt. % carbohydrate; and
wherein hydrolysed protein and intact protein together are present in a concentration (w/w) that exceeds the carbohydrate concentration (w/w).

21. A method of preventing and/or treating lipid metabolism disorders in humans, said method comprising ingesting a composition containing, calculated on dry matter:
10-100 wt % protein hydrolysate originating from a non-mammalian source;
0-90 wt. % intact protein;
0-50 wt. % carbohydrate; and
wherein hydrolysed protein and intact protein together are present in a concentration (w/w) that exceeds the carbohydrate concentration (w/w).

22. A cosmetic method of stimulating and/or improving body weight reduction in order to improve the human body appearance, said method comprising ingesting a composition containing, calculated on dry matter:
10-100 wt % protein hydrolysate originating from a non-mammalian source;
0-90 wt. % intact protein;
0-50 wt. % carbohydrate; and
wherein hydrolysed protein and intact protein together are present in a concentration (w/w) that exceeds the carbohydrate concentration (w/w).

23. The method of claim 20, wherein the composition contains the protein hydrolysate and the optional carbohydrate in a weight ratio of more than 1:3.

24. The method of claim 20, wherein the composition contains the protein hydrolysate and the optional carbohydrate in a weight ratio of more than 1:1.

25. The method of claim 20, wherein the composition contains 0-30% carbohydrate by weight of dry matter.

26. The method of claim 20, wherein the composition contains 20-80% intact protein by weight of dry matter.

27. The method of claim 20, wherein the composition contains between 0.5 and 5% rapidly and/or moderately absorbed carbohydrates by weight of dry matter and optionally contains slowly absorbed carbohydrates.

28. The method of claim 27, wherein the rapidly absorbed carbohydrates are selected from glucose, sucrose, maltose, and mixtures thereof, the moderately absorbed carbohydrates are selected from fructose, mannose, maltodextrin, white flour, wheat flour, and mixtures thereof, and the slowly absorbed carbohydrates are selected from raw corn starch, high amylose corn starch, modified starch, and mixtures thereof.

29. The method of claim 27, wherein the composition contains slowly absorbed carbohydrates in a concentration (w/w) that exceeds the combined concentration (w/w) of rapidly and moderately absorbed carbohydrates by at least a factor 2.

30. The method of claim 20, wherein the composition is selected from the group consisting of beverages, snacks and soups.

31. The method of claim 20, comprising ingesting the composition between 0.5 and 2 hours prior to the next meal.

32. The method of claim 20, comprising ingesting the composition in an amount equivalent to at least 5 g, preferably at least 10 g of the combination of protein hydrolysate and intact protein per serving.

33. The method of claim 20, wherein the protein hydrolysate is a vegetable protein hydrolysate, preferably a hydrolysate of a vegetable protein selected from the group consisting of pea protein, rice protein, soy protein and combinations thereof.

34. A nutritional beverage containing:
0.5-10 wt. % hydrolysed protein from a non-mammalian source;
0-10 wt. % intact protein;
0.5-5 wt. % carbohydrates; artificial sweetener; flavouring; and at least 80 wt. % water;
wherein hydrolysed protein and intact protein together are present in a concentration (w/w) that exceeds the carbohydrate concentration (w/w).

35. Nutritional beverage according to claim 34, wherein hydrolysed protein and intact protein together represent at least 5 wt. % of the beverage.

36. A savoury snack product containing:
3-50 wt. % hydrolysed protein from a non-mammalian source;
10-80 wt. % intact protein;
0-50 wt. % carbohydrate;
0.3-3 wt. % salt; and
0.1-20 wt. % water;
wherein hydrolysed protein and intact protein together are present in a concentration (w/w) that exceeds the carbohydrate concentration (w/w).

37. Snack product according to claim 36, wherein the product contains 1-20 wt. % dietary fibre.

38. A soup containing:
0.5-20 wt. % hydrolysed protein from a non-mammalian source;
0.5-29 wt. % intact protein;
0-20 wt. % carbohydrate;
0.1-2 wt. % salt; and
at least 70 wt. % water;
wherein hydrolysed protein and intact protein together are present in a concentration (w/w) that exceeds the carbohydrate concentration (w/w).
